(19) United States
(12) Patent Application Publication
(10) Pub. No.: US 2005/0227883 A1
(43) Pub. Date: Oct. 13, 2005

(54) Odor-eliminating hand scrub

(76) Inventor: Theresa M. Hogan, Andover, MN (US)

Correspondence Address: WALTER K. ROLOFF, 490 HARBOR COURT, SHOREVIEW, MN, 55126, US

(21) Appl. No.: 10/820,668

(22) Filed: Apr. 8, 2004

(51) Int. Cl. A61K 7/02

(52) U.S. Cl. 510/136

(57) ABSTRACT

An odor-eliminating hand scrub includes from one part to twenty-four parts by volume, at room temperature, of magnesium sulfate, and from one part to eight parts by volume, at room temperature, of a carrier oil.

### FIELD OF THE INVENTION

The present invention relates generally to skin cleansing products. The invention relates specifically to an odor-eliminating hand scrub for use after handling odoriferous foodstuffs, that is both environmentally-friendly and non-irritating.

### BACKGROUND OF THE INVENTION

The art of cooking and creating culinary delights has been, and continues to be, extremely popular. The many varieties of restaurants, cookware shops, cooking classes, and even television productions centered exclusively on cooking, indicate the popularity of this art.

Those who practice the art of cooking, from world-renowned chefs down through relatively unskilled young persons, generally enjoy working “in the kitchen”. Nonetheless, such work can sometimes be unpleasant. Chief among the un-pleasantries involves handling odoriferous foodstuffs such as, for example, cheese, fish, onions, and particularly garlic. As is well known, the strong and lingering effects of such odoriferous foodstuffs may be extremely difficult to remove; their strong odors linger on hands which handled them, even after aggressive washing of the hands with soap and water and other hand cleaning agents, such as environmentally-unfriendly detergents and surfactants. Often, the aggressive washing leads to skin irritation and the handler, or cook, is left with chapped, irritated hands which still emanate a strong, unpleasant odor.

Therefore, it would be desirable to provide an odor-eliminating hand scrub which (i) quickly and effectively removes odors from hands which have handled odoriferous foodstuffs, (ii) is environmentally-friendly, and (iii) aids in preventing hand chapping and irritation.

### SUMMARY OF THE INVENTION

An object of the present invention is to provide an odor-eliminating hand scrub which quickly and effectively removes odors from hands which have handled odoriferous foodstuffs.

Another object of the present invention is to provide an odor-eliminating hand scrub which is environmentally-friendly.

A further object of the present invention is to provide an odor-eliminating hand scrub which aids in preventing hand chapping and irritation.

In accordance with the present invention, an odor-eliminating hand scrub includes from one part to twenty-four parts by volume, at room temperature, of magnesium sulfate, and from one part to eight parts by volume, at room temperature, of a carrier oil.

### DETAILED DESCRIPTION OF THE INVENTION

As used here throughout, the phrase “odor-eliminating” is intended to include all compositions which are capable of substantially removing, masking, noticeably reducing, or eliminating odors. Also, the phrase “hand scrub” is intended to include all compositions for exfoliating, washing, scrubbing, or otherwise cleaning hands.

In a preferred embodiment of the odor-eliminating hand scrub of the present invention, the following ingredients are mixed together at room temperature:
(i) about ¾ cup (0.75 cup) of magnesium sulfate;
(ii) about ⅔ cup (0.67 cup) of a carrier oil; and
(iii) about 31 drops of essential or fragrance oils. The mixing of these ingredients is accomplished by stirring them together at room temperature in a suitably large mixing bowl utilizing a conventional stirring spoon, until the composition is well blended (as determined by visual appearance). Such aforedescribed blending yields slightly more than about 1.42 cups of the hand scrub of the present invention.

The aforementioned magnesium sulfate is commercially available as a conventional Epsom salt. Likewise, the aforementioned carrier oil and essential oils are also commercially available. The carrier oil is preferably extra virgin olive oil, but may also be olive oil, corn oil, sesame seed oil, sunflower seed oil, flax seed oil, or any other suitable, bio-degradable or organic oil. The essential or fragrance oils, in a preferred formulation embodiment, comprise a total of about 24 drops, as follows: about 16 drops of lavender oil; about 6 drops of grapefruit oil; and about 2 drops of basil oil.

In the foregoing description of the preferred embodiment, and for reference below, the proportions of about 0.75 cup of magnesium sulfate to about 0.67 cup of the carrier oil is approximately equivalent to a ratio of 9 parts of magnesium sulfate (MS) to 8 parts of carrier oil (CO) (i.e., 9MS:8CO). The relative proportion of essential oils is as aforestated.

In a preferred method of use of the hand scrub of the present invention, a user stirs the composition until it is again well-blended, since some settling and separation of the ingredients may occur between uses. The user then pours about one teaspoon of the composition into the hands. The user then massages or works the composition into and about the hands (i.e., scrubs) for about 45 seconds to 1 minute. The user finally rinses the composition from the hands with lukewarm (i.e., tepid or moderately warm) water.

Turning, again, to formulation of the hand scrub of the present invention, the following table illustrates exemplary ingredient proportional ranges and noted effects in accordance therewith:

| Ingredient Ratio | Effects |
|---|---|
| 1MS:8CO | removes odor; hands are oily. |
| 2MS:8CO | removes odor; hands are oily. |
| 3MS:8CO | removes odor; hands are soft with a thin oily film. |
| 4MS:8CO | removes odor; hands are soft with a thin oily film. |
| 5MS:8CO | removes odor; hands are soft with a thin oily film. |
| 6MS:8CO | removes odor; hands are soft and not oily. |
| 7MS:8CO | removes odor; hands are soft and not oily. |
| 8MS:8CO | removes odor; hands are soft and not oily. |
| 9MS:8CO | removes odor; hands are soft and not oily. |

The following controls were used relative to each of the foregoing trials: ingredients were blended in a non-porous container and were at about the same (room) temperature; a fresh cut of garlic was applied to the hands; a 45-second hand scrubbing procedure was utilized; and lukewarm tap water was used to rinse the hands after each successive garlic application and scrubbing.

Although not listed in the table, it was found that utilization of a ratio of up to 24MS:1CO still provided an effective way to remove odor; however, such higher concentrations of MS to CO resulted in a “harsh feeling” hand scrub.

It is believed that the novel odor-eliminating property of the present invention is achieved by way of an “active ingredient” of the composition, namely the magnesium sulfate in two respects. First, magnesium sulfate, MgSO₄-7H₂O, has a strong affinity for carbon. Since most odor-causing substances (plants and animals) comprise carbon to a large extent, the externally present magnesium sulfate binds with the carbon of the odoriferous substances and is then easily washed away. Second, with the skin of the hands being a semi-permeable osmotic membrane, the magnesium sulfate acts upon the skin to balance osmolarity therethrough. In this regard, the concentration of magnesium sulfate on the hands, upon application of the composition, is much higher relative to the concentration of magnesium sulfate occurring naturally within the skin of the hands. The skin of the hands, then, being a semi-permeable osmotic membrane, promotes fluid outflow to balance or equalize the osmolarity on each side of the membrane, thereby tending to balance the relative concentrations of magnesium sulfate internally and externally. This tendency or action effects a “drawing out” of the odoriferous substance from the hands, which may then be rinsed away.

It is to be particularly appreciated and understood that unlike conventional and well-known detergents, surfactants, and scrubs, the present invention is environmentally friendly since the aforementioned ingredients of the composition are natural and bio-degradable.

It is also to be appreciated and understood that the preferred formulation of the invention including the carrier oil provides protection against irritation from the hand scrubbing process, and additionally promotes skin smoothness. Use of extra virgin olive oil as the carrier oil has been found to be particularly advantageous in this regard.

In formulation of the environmentally-friendly, odor-eliminating hand scrub of the present invention, it is to be understood that various suitable components may be substituted for those described, or some components may even be omitted. Thus, a suitable oil not specifically listed herein may serve as the carrier oil. Also, an alternative embodiment of the present invention may simply omit the aforementioned fragrance oils without diminishing the odor-eliminating properties.

It is also to be appreciated and understood that the environmentally-friendly, odor-eliminating hand scrub of the present invention has been found to provide other uses outside the cooking arts. For example, the hand scrub is beneficial to: veterinarians and animal-care professionals who are subject to strong odors from handling animals and clinical liquids (e.g., iodine and antiseptics) during veterinary procedures; fish and seafood handlers (e.g., commercial fishermen, seafood processing and cannery workers, and recreational anglers); livestock (e.g., swine and cattle) handlers; those working in a natural environment (e.g., forest rangers, conservation officials, surveyors, naturalists, and law enforcement officials) where unpleasant odors may be encountered (e.g., from handling decaying organic and animal matter); and smokers who are subject to nicotine odors from handling cigarettes.

Further, it has been found that use of the hand scrub of the present invention, when followed by a conventional mild soap wash, eliminates gasoline odor. This is particularly useful to mechanics, machinists, and others who handle odoriferous petroleum-based liquids, compositions, and solvents.

With additional regard to petroleum products, it is also to be appreciated and understood that the environmentally-friendly, odor-eliminating hand scrub of the present invention has been found to effectively remove oil-based paint from skin (with “paint” collectively referring to paints and other pigmented coatings) without irritation, according to the same application protocol as aforedescribed.

Although the instant disclosure has been directed to human hands, it is to be appreciated that the hand scrub of the present invention could advantageously be used with regard to any human or animal skin portion that is similar to the skin of human hands.

While the present invention has been particularly described with reference to certain chemical compositions, it will be understood, however, that other modifications thereto are of course possible, all of which are intended to be within the true spirit and scope of the present invention. It should be appreciated that components of the invention aforedescribed may be substituted for other suitable components for achieving desired results, or that various accessories may be added thereto.

Lastly, the choice, of course, of compositions, sizes, and values of various aforementioned components of the present invention are all a matter of design choice depending upon intended uses thereof.

Accordingly, these and other various changes or modifications in form and detail of the present invention may also be made therein, again without departing from the true spirit and scope of the invention as defined by the appended claims.

1. An odor-eliminating hand scrub for use after handling odoriferous foodstuffs, comprising:
(i) from one part to twenty-four parts by volume, at room temperature, of magnesium sulfate; and
(ii) from one part to eight parts by volume, at room temperature, of a carrier oil.

2. The odor-eliminating hand scrub of claim 1, wherein said magnesium sulfate is Epsom salt.

3. The odor-eliminating hand scrub of claim 1, wherein said carrier oil is selected from the group consisting of extra virgin olive oil, olive oil, corn oil, sesame seed oil, sunflower seed oil, and flax seed oil.

4. The odor-eliminating hand scrub of claim 1, further comprising from about one to twenty-four drops by volume, at room temperature, of at least one essential oil.

5. The odor-eliminating hand scrub of claim 4, wherein said at least one essential oil is selected from the group consisting of lavender oil, grapefruit oil, and basil oil.

6. The odor-eliminating hand scrub of claim 4, wherein said magnesium sulfate is Epsom salt.

7. The odor-eliminating hand scrub of claim 4, wherein said carrier oil is selected from the group consisting of extra virgin olive oil, olive oil, corn oil, sesame seed oil, sunflower seed oil, and flax seed oil.

8. An odor-eliminating hand scrub for use after handling odoriferous foodstuffs, comprising:
(i) about nine parts by volume, at room temperature, of magnesium sulfate; and
(ii) about eight parts by volume, at room temperature, of a carrier oil.

9. The odor-eliminating hand scrub of claim 8, wherein said magnesium sulfate is Epsom salt.

10. The odor-eliminating hand scrub of claim 8, wherein said carrier oil is selected from the group consisting of extra virgin olive oil, olive oil, corn oil, sesame seed oil, sunflower seed oil, and flax seed oil.

11. The odor-eliminating hand scrub of claim 8, further comprising about twenty-four drops by volume, at room temperature, of at least one essential oil.

12. The odor-eliminating hand scrub of claim 11, wherein said at least one essential oil is selected from the group consisting of lavender oil, grapefruit oil, and basil oil.

13. The odor-eliminating hand scrub of claim 11, wherein said magnesium sulfate is Epsom salt.

14. The odor-eliminating hand scrub of claim 11, wherein said carrier oil is selected from the group consisting of extra virgin olive oil, olive oil, corn oil, sesame seed oil, sunflower seed oil, and flax seed oil.

15. A method of eliminating a strong foodstuff odor emanating from hands, comprising the steps of:
providing an odor-eliminating hand scrub;
stirring said hand scrub until it is well-blended;
applying about one teaspoon of said hand scrub to hands emanating said strong odor;
massaging said hand scrub into hands for about forty-five seconds to one minute; and
rinsing the hands with lukewarm water.

16. The method of claim 15, for removing oil-based paint from hands.
